(19) United States
(12) Patent Application Publication
(10) Pub. No.: US 2026/0216533 A1
(43) Pub. Date: Jul. 30, 2026

(54) PATIENT COUCH FOR POSITIONING AND/ OR SUPPORTING A PATIENT DURING RADIATION THERAPY

(71) Applicant: Varian Medical Systems, Inc., Palo Alto, CA (US)

(72) Inventors: Patrick PLANNERER, Kulmain (DE); Markus BARTHEL, Kemnath (DE); Andreas BUCHENAUER, Bayreuth (DE); Klaus HRUSCHKA, Erbendorf (DE); Berthold BAUMANN, Kastl (DE); Wolfgang NEUBER, Eschenbach i. d. Opf. (DE); Thomas SCHMAELZLE, Bayreuth (DE); Daniel EIGNER, Erbendorf (DE); Konstantin KNODT, Erbendorf (DE); Stefan BUCHBINDER, Speichersdorf (DE); Katrin KILIAN, Weiden (DE); Johannes STOCK, Reuth bei Erbendorf (DE)

(73) Assignee: Varian Medical Systems, Inc., Palo Alto, CA (US)

(21) Appl. No.: 19/457,266

(22) Filed: Jan. 23, 2026

(30) Foreign Application Priority Data
Jan. 24, 2025 (EP) 25153793.2

(51) Int. Cl. A61N 5/10

(52) U.S. Cl. CPC A61N 5/1048

(57) ABSTRACT

One or more example embodiments relates to a patient couch for positioning and/or supporting a patient for radiotherapy, comprising a couch board section to accommodate the patient; a base section attachable to a floor; a roll-pitch assembly between the base section and the couch board section, the roll-pitch assembly configured to pivot the couch board section about at least one of a roll axis or a pitch axis; and a cover unit forming a shell cover and comprising at least two cover shells, wherein one of the at least two cover shells forms a roll cover shell and another of the at least two cover shells forms a pitch cover shell, the roll cover shell is configured to compensate a roll movement of the roll-pitch assembly, and the pitch cover shell is configured to compensate a pitch movement of the roll-pitch assembly.

### CROSS-REFERENCE TO RELATED APPLICATION(S)

The present application claims priority under 35 U.S.C. § 119 to European Patent Application No. 25153793.2, filed Jan. 24, 2025, the entire contents of which are incorporated herein by reference.

### FIELD

Independent of the grammatical term usage, individuals with male, female or other gender identities are included within the term.

One or more example embodiments relates to a patient couch for positioning and/or supporting a patient during radiation therapy. The patient couch comprises a couch board section for accommodating the patient, a base section for attachment to the floor, a roll-pitch assembly arranged between the base section and the couch board section, designed to pivot the couch board section about a roll axis and/or a pitch axis.

### RELATED ART

Patient couches are widely used in various medical fields for the transport and positioning of patients during imaging, treatment, and/or inpatient care. Additionally, patient couches play a critical role in radiation therapy, where precise positioning and stability are essential to ensure accurate treatment delivery.

Patient couches employed in radiation therapy must be particularly robust against radiation and enable highly precise positioning of the patient. This is critical to ensure that the radiation is accurately directed at the target area while sparing the surrounding healthy tissue as much as possible. In radiation therapy, it is customary for patient couches to be designed to offer high stability and precision. The couches must be capable of holding the patient in a fixed position to minimize movement during treatment. This is often achieved through the use of fixation devices and specialized positioning aids. For patient positioning, the couches include mechanisms for horizontal and vertical adjustments as well as mechanisms for tilting (pitch, roll).

The mechanisms for tilting, swiveling, and positioning the patient must be designed to prevent accidental interference. This is important to avoid pinching injuries to hands, patients, or staff. Safety covers and protective devices are therefore integral parts of the design to minimize the risk of injury. To prevent such interferences covers may be used.

Covers are used also for aesthetic reasons but, more importantly, for regulatory compliance. These covers must be particularly easy to clean, hygienic, and suitable for use as medical devices. At the same time, they must be resistant to harsh ionizing radiation to ensure their functionality and integrity throughout the product's lifetime.

Covers are used also for aesthetic reasons but, more importantly, for regulatory compliance. These covers must be particularly easy to clean, hygienic, and suitable for use as medical devices. At the same time, they must be resistant to harsh ionizing radiation to ensure their functionality and integrity throughout the product's lifetime. Moreover, the covers should be designed for easy assembly and disassembly to facilitate maintenance and service, and their movement should be smooth and quiet, without any abnormal noises.

### SUMMARY

Patient couches in the prior art often impose significant constraints on the available treatment volume, which can hinder the effectiveness and flexibility of radiation therapy procedures. This limitation is primarily caused by physical collisions between the couch covers and the gantry, or between the couch covers and the gantry covers, particularly when the couch is adjusted for specific roll and pitch positions. Such collisions not only restrict the range of possible patient positioning but may also disrupt the smooth operation of the equipment. Additionally, existing covers for roll-pitch-capable couches are frequently bulky, adding unnecessary complexity to the system. They are also often difficult to clean, which poses challenges for maintaining hygiene standards, especially in a clinical environment where strict sanitation is required.

One or more example embodiments provides a patient couch for use in radiation therapy that addresses these issues. The goal is to develop a couch that significantly improves upon the limitations of the prior art, offering enhanced compatibility with treatment equipment and expanded positioning flexibility. Moreover, one or more example embodiments creates a solution that incorporates a more compact and streamlined design for the covers, ensuring ease of cleaning and compliance with stringent hygiene requirements, while maintaining robustness against the effects of ionizing radiation.

### BRIEF DESCRIPTION OF THE DRAWINGS

Embodiments, functionalities, and effects of example embodiments are apparent from the accompanying figures and their descriptions. The figures illustrate:

FIG. 1 illustrates a radiotherapy device with a patient couch according to one or more example embodiments;

FIG. 2 illustrates a section of the patient couch, comprising a roll-pitch-assembly according to one or more example embodiments;

FIGS. 3a, 3b, 3c, and 3d illustrate the roll-pitch assembly after different roll-and/or pitch movements according to one or more example embodiments;

FIG. 4 illustrates an embodiment of the cover unit; and

FIG. 5 a-i illustrate a section of the patient couch for various roll and/or pitch movements according to one or more example embodiments.

### DETAILED DESCRIPTION

The patient couch is configured for positioning and/or supporting a patient or an object for a radiotherapy. The patient couch is preferably stationary. Alternatively, the patient couch can be mobile. The patient can be positioned on the patient couch, particularly in a lying position. Specifically, the patient couch includes means for securing and/or fixing the patient. Examples of such means include straps, clamps, and customized cushions that conform to the patient's body shape to ensure stability and comfort during treatment.

The patient couch can be part of a radiotherapy system and/or can be used or positioned near the treatment area of a LINAC (Linear Accelerator). The patient couch is particularly designed for height adjustment, allowing the patient to be lowered or raised as needed. Furthermore, the patient couch is preferably designed to move the patient and/or the couch board section horizontally, for example, in two orthogonal directions, such as longitudinal and transverse directions.

This horizontal movement capability is crucial for precise positioning, ensuring that the patient is accurately aligned with the radiation beam. The combination of vertical and horizontal adjustments allows for fine-tuning the patient's position, which is essential for targeting the treatment area while minimizing exposure to surrounding healthy tissues.

The patient couch comprises a couch board section designed to accommodate the patient. This section, often referred to as the patient support board or includes a patient support board. The couch board section, especially the patient support board, is configured for providing a stable and comfortable surface for the patient during radiotherapy treatments. couch support boards can be made from various materials, each chosen for its specific properties. Preferably the couch board section, especially the patient support board, is based on carbon fiber composites, which provide strength, rigidity, and lightweight. Carbon fiber is particularly advantageous in radiotherapy settings because it is radiolucent, meaning it does not interfere with the radiation beams, allowing for precise targeting of the treatment area. Furthermore, couch board section, especially the patient support board, can be based or made of high-density polyethylene and specialized medical-grade plastics, which offer durability and ease of cleaning.

The couch board section, especially the patient support board, is constructed to ensure minimal deflection and maximum stability. They couch board section, especially the patient support board, preferably features a modular design, allowing for easy attachment of accessories such as immobilization devices and positioning aids. This modularity is essential for customizing the setup to meet the specific needs of each patient and treatment plan.

Preferably, the couch board section, especially the patient support board, comprises an indexing systems that allow for reproducible positioning, ensuring that the patient can be placed in the exact same position for each treatment session. The couch board section, especially the patient support board, can comprise markers, especially radiopaque markers, for positioning and/or position verification. Optionally, the couch board section may have built-in channels or slots for securing straps and other fixation devices, enhancing patient stability and safety during treatment.

The patient couch includes a base section designed for attachment to the floor. This base section is configured for providing stability and support for the entire couch structure. It can be constructed to form a secure connection with the floor, ensuring that the couch remains stationary during patient positioning and treatment.

The base section can serve as a foundation or pedestal for the patient couch. It may include a lifting mechanism, such as a scissor lift, to enable vertical adjustment of the patient couch. This allows the patient to be raised or lowered to the desired height, facilitating precise alignment with the radiation beam.

To enhance safety and functionality, the base section is preferably enclosed. This enclosure protects the internal mechanisms from dust and debris, and also prevents accidental contact with moving parts, reducing the risk of injury to patients and medical staff. The enclosure can be made from durable materials that are easy to clean and disinfect, ensuring compliance with medical hygiene standards. Preferably, the base section may incorporate features for horizontal movement, allowing the patient couch to be shifted in two orthogonal directions, such as longitudinal and transverse directions.

The design of the base section also takes into account the need for robustness and durability. It must be capable of supporting the weight of the patient and any additional equipment used during treatment. The materials used in its construction should be resistant to wear and tear, as well as to the effects of ionizing radiation, ensuring long-term reliability and safety.

The patient couch includes a roll-pitch assembly positioned between the base section and the couch board section. The a roll-pitch assembly is designed to pivot the couch board section about a roll axis and/or a pitch axis, providing precise adjustments to the patient's position during radiotherapy. The roll-pitch assembly allows for the rotation of the couch board section around two primary axes: the roll axis and the pitch axis. This capability allows achieving optimal patient alignment with the radiation beam, ensuring accurate targeting of the treatment area while minimizing exposure to surrounding healthy tissues.

The roll-pitch assembly can be constructed using various mechanical components, such as bearings, actuators, and control systems. These components work together to provide smooth and precise movements. The a roll-pitch assembly may include motorized actuators that enable automated adjustments, controlled via a user interface or integrated software.

Preferably, the roll-pitch assembly is configured as a cardanic mechanism and/or a gimbal mechanism. For example, the roll-pitch assembly involves the use of a gimbal mechanism. The roll-pitch assempbly may comprise two intersecting and orthogonally arranged rotational axes. This gimbal setup, also known as a Cardanic suspension, allows for independent rotation around both the roll and pitch axes. The gimbal mechanism ensures that the couch board section can be precisely tilted and rotated, providing enhanced flexibility in patient positioning.

The patient couch comprises a shell cover and comprises at least two cover shells, wherein one of the cover shells forms a roll cover shell and another of the cover shells forms a pitch cover shell. The roll cover shell is designed and/or arranged to compensate for the roll movement of the roll-pitch assembly, ensuring that the roll-pitch assembly is covered in all roll settings.

Similarly, the pitch cover shell is designed and/or arranged to compensate for the pitch movement of the roll-pitch assembly, ensuring that the roll-pitch assembly is covered in all pitch settings. The shell cover serves multiple purposes, including concealing the roll-pitch assembly and providing pinch protection for hands. This is crucial for ensuring the safety of both patients and medical staff, preventing accidental injuries during adjustments.

The cover shells are typically made from durable, lightweight materials, for example such as high-density polyethylene (HDPE) or medical-grade plastics. These materials are chosen for their strength, ease of cleaning, and resistance to wear and tear. Additionally, they must be resistant to ionizing radiation to maintain their integrity and functionality in a radiotherapy environment.

The cover shells are preferably configured as hard cover cases. Favorably, the cover shells are resistant to ionizing radiation and/or invisible in X-ray images. In particular, the cover shells are based on and/or made of carbon fiber composites, polycarbonates, Boron Nitride Nanotubes (BNNTs), Polyetherimid (PEI), High-Density Polyethylene (HDPE), Polyetheretherketon (PEEK), Polyimid (PI), Polyamid (PA), Polysulfon (PSU) and/or graphites. These materials ensure that the hard cover cases are both durable and suitable for use in environments with ionizing radiation, while also being compatible with X-ray imaging requirements. It's also possible to use other materials, such as polystyrene produced through the low-pressure process or polyamide with fiber-reinforced inserts produced through the high-pressure process.

In particular, the roll cover shell and pitch cover shell are designed to move in coordination with the roll-pitch assembly. This ensures that the assembly remains covered regardless of its position. Optionally, the roll cover shell compensates for the roll movement by rotating along with the roll axis, while the pitch cover shell compensates for the pitch movement by tilting along with the pitch axis. The cover shells can be fixed to the roll-pitch assembly with flexible joints that allow for smooth movement, ensuring that the shells move seamlessly with the assembly, providing continuous coverage. A prefered configuration involves telescopic shells that can extend and retract as needed, allowing the shells to adjust their length to cover the assembly fully, regardless of its position. The cover shells can also be segmented, with each segment capable of independent movement, allowing for more precise adjustments and ensuring that the assembly is always covered, even during complex movements.

The roll cover is designed to adjust dynamically during a roll and/or pitch movement of the roll-pitch assembly. For a roll movement it can involve a mechanism where the roll cover shortens on one side or partially slides into another cover element and/or shell, while on the opposite side, it extends or deploys to maintain continuous coverage of the roll-pitch assembly.

Especially, during a roll movement, the roll cover shell on the side that is moving downward will retract. This retraction can be achieved through a telescopic design, where the cover segments and/or shells slide into each other, or through a flexible joint mechanism that allows the cover to fold or collapse. On the opposite side, the roll cover will extend to compensate for the upward movement. This extension can be facilitated by a spring-loaded mechanism, a motorized actuator that pushes the cover outward or by connecting the cover parts to the moving frames, ensuring that the roll-pitch assembly remains fully covered. The pitch movement can involve an analogous and/or similar mechanism.

The shell cover is designed to meet stringent safety and hygiene standards. It provides a barrier that prevents accidental contact with the moving parts of the roll-pitch assembly, reducing the risk of injury. The materials used are easy to clean and disinfect, ensuring that the patient couch remains hygienic and suitable for medical use. Overall, the shell cover with its roll and pitch cover shells is a critical component of the patient couch, providing safety, functionality, and durability in a radiotherapy setting.

Preferably, the cover shells are movable relative to each other in the vertical direction. This feature allows the cover shells to adjust their position to accommodate the movements of the roll-pitch assembly, ensuring continuous coverage and protection. For example, the cover unit and/or cover shells can be designed to telescope into each other. In this configuration, the cover unit consist of multiple cover shells as segments that can slide into one another, similar to the sections of a telescope.

While the cover shells are movable relative to each other in the vertical direction, they are in particular fixed relative to each other in the horizontal plane. This means that the cover shells maintain their alignment and position horizontally, ensuring stability and preventing any lateral movement that could compromise the coverage or protection of the roll-pitch assembly.

The cover shells are preferably designed to be slidable into and out of each other to compensate for a pitch and/or roll movement. This feature allows the cover shells to dynamically adjust their position, ensuring continuous coverage of the roll-pitch assembly during its movements. For instance, in the case of a pitch movement, the cover shells can slide vertically relative to each other. When the roll-pitch assembly tilts forward or backward, the cover shells on one side can retract by sliding into the adjacent cover shell, while the cover shells on the opposite side extend by sliding out. This telescopic mechanism ensures that the roll-pitch assembly remains covered at all times, regardless of its pitch angle. Similarly, for a roll movement, the cover shells can slide horizontally relative to each other. As the roll-pitch assembly tilts to the left or right, the cover shells on the descending side can retract by sliding into the adjacent cover shell, while the cover shells on the ascending side extend by sliding out. This ensures that the roll-pitch assembly is continuously covered during the roll movement.

Preferably, the cover shells are designed to be receivable into each other to compensate for a pitch and/or roll movement. This means that the cover shells can nest within each other, providing a compact and efficient way to adjust their length. For example, during a pitch movement, the cover shells on one side can nest into the adjacent cover shell, reducing their overall length, while the cover shells on the opposite side extend by sliding out. This nesting mechanism ensures that the cover shells can adjust their length dynamically, providing continuous coverage of the roll-pitch assembly. Furthermore the covers can be designed in such a way that liquids run off from the top downwards and do not enter the interior.

Optionally, at least one of the cover shells has a projection, and a cover shell that is received by or receives the cover shell has a receiving section corresponding to the projection. The projection and the receiving section form a guidance system and/or interlock in such a way that the vertical movement is limited. The projection on one cover shell can be designed to fit precisely into the receiving section of the adjacent cover shell. This mechanism ensures that the cover shells move together in a controlled manner, providing stability and preventing unintended vertical displacement. The guidance system formed by the projection and the receiving section allows for smooth and precise adjustments, ensuring that the cover shells maintain continuous coverage of the roll-pitch assembly during its movements.

The projection can be a ridge or a rail that extends along the edge of the cover shell, while the receiving section can be a corresponding groove or channel on the adjacent cover shell. When the cover shells are assembled, the projection slides into the receiving section, creating a secure interlock that guides the vertical movement. This design ensures that the cover shells move in unison, maintaining their alignment and preventing any gaps or misalignment that could compromise the coverage of the roll-pitch assembly. The materials used for the projection and receiving section are typically the same as those used for the cover shells, such as high-density polyethylene (HDPE) or medical-grade plastics. These materials provide the necessary strength and durability for the interlocking mechanism, ensuring long-term reliability and functionality.

In a preffered embodiment, one of the cover shells forms an intermediate cover shell, wherein the intermediate cover shell is arranged between the roll cover shell and the pitch cover shell. The intermediate cover shell is designed and/or arranged to compensate for a roll and a pitch movement at least partially. The intermediate cover shell serves as a transitional element that bridges the roll cover shell and the pitch cover shell. This intermediate cover shell is for ensuring continuous coverage and smooth movement of the cover system during both roll and pitch adjustments. By being positioned between the roll cover shell and the pitch cover shell, the intermediate cover shell can adapt to the combined movements of the roll-pitch assembly, providing flexibility and stability. The intermediate cover shell is preferably designed to move in coordination with both the roll and pitch cover shells. It can slide or telescope into and out of the adjacent cover shells, allowing it to extend or retract as needed to accommodate the movements of the roll-pitch assembly.

Particularly, the roll-pitch assembly comprises a base frame and a pitch frame, wherein the base frame is fixedly connected to the base section and/or rests on the base section. The pitch frame is pivotable about the pitch axis, wherein the pitch axis rests on the base frame and/or the pitch axis is guided through an opening of the base frame. The base frame and pitch frame form a structural framework, which can be configured as a frame, box, or chassis. These frames are designed to be rigid and stable, providing a solid foundation for the roll-pitch assembly. They can be constructed from materials such as high-strength plastics or metals, including aluminum or steel, to ensure durability and resistance to mechanical stress. The axes, particularly the pitch axis, are designed as rotational axes. These axes are mechanical components that pass through specified parts of the frames, such as openings or bores. The pitch axis, for example, may be guided through an opening in the base frame, allowing the pitch frame to pivot smoothly around this axis. The axes can be supported by one or more bearings, which facilitate smooth and controlled rotational movement. These bearings can be ball bearings, roller bearings, or other types suitable for the specific mechanical requirements of the roll-pitch assembly.

The base frame is preferably securely attached to the base section of the patient couch, either through fixed connections such as bolts or welding, or by resting on the base section with a stable interface. This ensures that the base frame remains stationary and provides a reliable support structure for the pitch frame. The pitch frame, pivotable about the pitch axis, allows for precise adjustments of the couch board section's angle. This pivoting mechanism is essential for achieving the desired patient positioning during radiotherapy. The pitch axis, guided through the base frame, ensures that the pitch frame can move freely and accurately, maintaining the stability and alignment of the entire assembly.

Preferably, the roll-pitch assembly comprises a roll frame, wherein the roll frame and the roll axis are pivotable. The roll axis rests on the pitch frame and/or is guided through an opening in the pitch frame so that the roll axis follows a pivoting of the pitch frame about the pitch axis. The roll frame forms a structural component that supports the roll axis and allows for its pivoting movement. This frame can be constructed from rigid materials such as high-strength plastics or metals, including aluminum or steel, to ensure durability and stability. The roll frame is designed to pivot around the roll axis, enabling the couch board section to tilt laterally. The roll axis, which is a rotational axis, is supported by the pitch frame. This means that the roll axis is either directly mounted on the pitch frame or passes through an opening in the pitch frame. The opening can be a precisely machined bore or slot that allows the roll axis to rotate smoothly. Bearings may be used to support the roll axis, providing low-friction movement and ensuring precise control over the pivoting action. These bearings can be ball bearings, roller bearings, or other suitable types, depending on the mechanical requirements.

Optionally, a mechanical connection of the cover unit is made via the top and bottom cover shell in the vertical direction, wherein the top cover shell is mechanically connected to the couch board section and the bottom cover shell is mechanically connected to the base section. The top cover shell is designed to move in unison with the couch board section. This mechanical connection ensures that any vertical adjustments of the couch board section are mirrored by the top cover shell, maintaining continuous coverage and protection of the roll-pitch assembly. The connection can be achieved through various means, such as brackets, hinges, or sliding mechanisms, which securely attach the top cover shell to the couch board section. These connections are designed to be robust and durable, capable of withstanding repeated movements without compromising the integrity of the cover unit. Similarly, the bottom cover shell is mechanically connected to the base section. This connection ensures that the bottom cover shell remains stationary relative to the base section, providing a stable foundation for the cover unit.

Especially, the cover shells are designed to be form-stable, providing a rigid and durable hard cover for the roll-pitch assembly. Mechanically, the cover shells are for example constructed from high-strength materials such as high-density polyethylene (HDPE) or medical-grade plastics, which ensure their structural integrity and resistance to deformation under stress.

One or more example embodiments is also related to a radiotherapy device comprises the patient, a control unit for controlling and/or adjusting the roll-pitch assembly, and a linear accelerator (LINAC) or a radiation source for generating therapeutic radiation. The patient couch is arranged and/or designed so that a patient can be positioned at a desired irradiation angle.

The LINAC is a component of the radiotherapy device, responsible for generating high-energy X-rays or electron beams used to treat cancerous tissues. The LINAC connected with a gantry, which is a rotating structure that allows the radiation source to move around the patient. This movement enables the delivery of radiation from multiple angles, ensuring precise targeting of the tumor while minimizing exposure to surrounding healthy tissues.

The patient couch, in combination with the LINAC, plays a vital role in patient positioning and treatment accuracy. The couch is designed to be highly adjustable, allowing for precise control of the patient's position. The roll-pitch assembly, controlled by the control unit, enables the couch to pivot around the roll and pitch axes, ensuring that the patient can be positioned at the exact angle required for effective irradiation. The control unit is responsible for managing the movements of the roll-pitch assembly, allowing for fine adjustments to the patient's position. This ensures that the patient remains in the correct position throughout the treatment, even if minor adjustments are needed during the session. The control unit can be integrated with the LINAC's control system, providing a seamless interface for the radiotherapy technician to manage both the radiation delivery and patient positioning.

One or more example embodiments relates to the use of the patient couch for positioning a patient at an adjustable irradiation angle in a radiotherapy device. The patient couch, equipped with a roll-pitch assembly and optinally controlled by a dedicated control unit, allows for precise adjustments of the patient's position. This ensures optimal alignment with the therapeutic radiation beam generated by a linear accelerator (LINAC) or other radiation sources. The patient couch's ability to pivot around roll and pitch axes, combined with its vertical and horizontal adjustability, enables accurate positioning at various irradiation angles. This enhances the effectiveness of radiotherapy treatments by allowing for targeted delivery of radiation to the tumor while minimizing exposure to surrounding healthy tissues. The integration of the patient couch with the radiotherapy device ensures seamless operation and improved patient outcomes.

Features disclosed in connection with particular embodiments of the invention may be combined with features of other embodiments, even if such combinations are not explicitly stated. In particular, features pertaining to different claim categories, such as methods, devices, and uses, are considered mutually transferable, provided that such a combination is technically feasible and does not deviate from the core inventive concept. These combinations are within the scope of the invention as disclosed herein.

The invention is not limited to the specific embodiments described herein. Variations, modifications, and alternative configurations that fall within the scope of the appended claims and are equivalent to the disclosed embodiments are also encompassed by the invention. Unless otherwise stated, terms used in the claims and description should not be interpreted in a restrictive manner, but rather in accordance with their broadest technically feasible meaning, as understood by a person skilled in the art.

One or more example embodiments aims to address one or more technical problems as identified herein or as may become apparent to a person skilled in the art. However, example embodiments are not limited to solving the explicitly stated problems but also encompasses any other advantages or improvements achieved through its implementation.

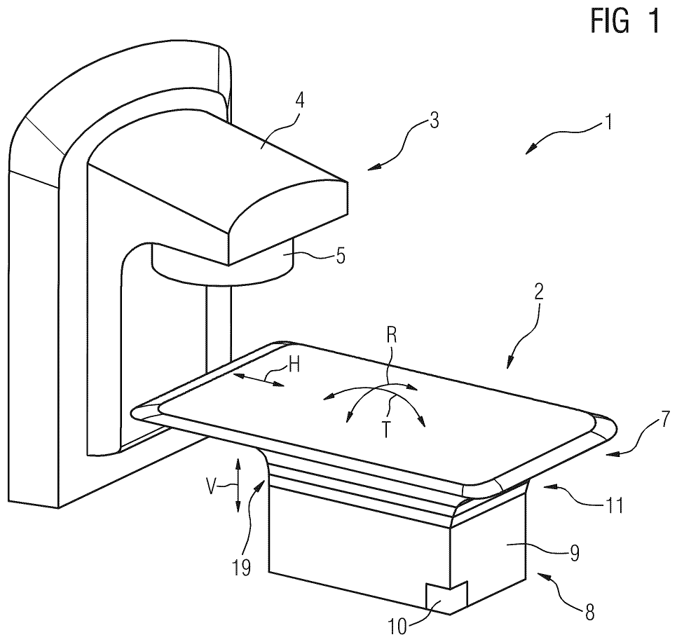

FIG. 1 shows a radiotherapy device 1. The radiotherapy device 1 comprises a patient couch 2 and a linear accelerator 3. The linear accelerator 3 includes a gantry 4 and a beam outlet 5. The linear accelerator 3 and the patient couch 2 are arranged such that a patient positioned on the patient couch 2 can be treated with radiation from the linear accelerator 3 during radiotherapy treatment.

The patient couch 2 comprises a couch board section 7 and a base section 8. The couch patient section 7, especially the upper part of the patient couch 2, provides accommodation and fixation for the patient. The couch board section 7 preferably includes a flat or curved surface and defines a horizontal plane. The surface of the couch board section 7 is preferably transparent to x-ray and high-energy photon beams.

The base section 8, particularly the lower part of the patient couch 2, connects the patient couch 2 to the ground and may include mechanisms for vertical V and horizontal H movement of the patient. The couch board section 8 include mechanisms for rotational R and tilting T movements, allowing optimal positioning of the patient for treatment. The base section 8 is preferably covered and/or shielded by a cover 9.

The radiotherapy device 1 and patient couch 2 include a control unit 10, configured to control and/or steer the rotational and tilting movements, as well as the horizontal and vertical movements of the patient couch 2. The control unit 10 may also be integrated with the control unit of the linear accelerator 3, ensuring coordinated positioning and treatment of the patient with radiation.

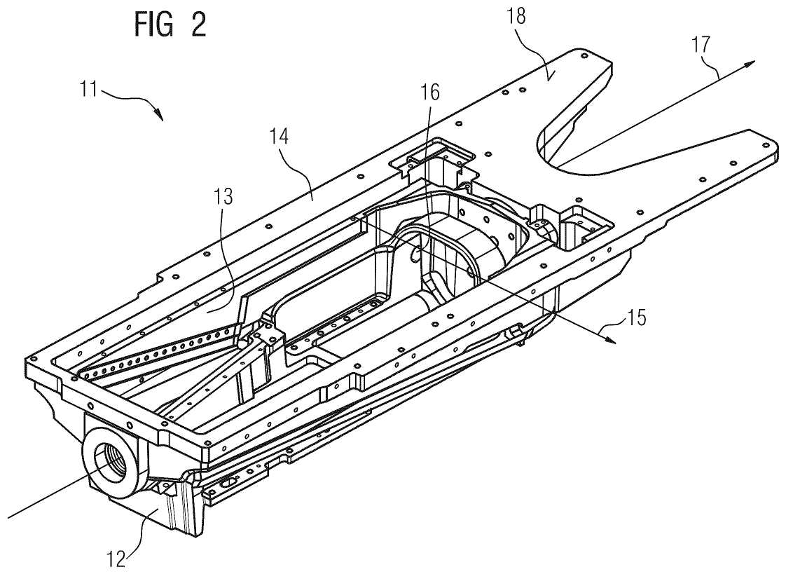

FIG. 2 shows a section of the roll-pitch assembly 11. The roll-pitch assembly 11 is positioned between the couch board section 7 and the base section 8. The depicted assembly is modular and includes a base frame 12, a pitch frame 13, and a roll frame 14. The frames 12 to 14 form rigid structures, typically made of metallic materials such as steel or made of plastics or graphite. The frames are partially nested within one another.

The base frame 12 forms the lowest section in the vertical direction V and is fixed and/or firmly mounted. The base frame 12 sits on the base section 8. In other words, the base frame 12 is connected to the floor through the base section 8. As part of a height adjustment mechanism, particularly through a mechanism in the base section 8, the base frame 12 can also be adjusted in height and/or longitutinal direction. The base frame 12, for example, forms a trapezoidal frame in top view. To minimize weight and improve radiation transparency, the edges of the frame are essentially solid, so that in top view, the interior of the frame is only reinforced by individual struts.

The pitch frame 13 at least partially surrounds the base frame 12. The pitch frame 13 is designed in the shape of a frame and is essentially rectangular in a top view. The pitch frame 13 is pivotable and/or rotatable about a pitch axis 15. The pitch axis 15 forms in particular a mechanical axis, for example a rod-shaped or cylindrical metal axis. The pitch frame 13 is firmly connected to the pitch axis 15, in particular in a material-locking manner, for example welded, form-locking, or force-locking.

The pitch axis 15 extends through the base frame 12 and/or is rotatably received or mounted on the base frame. For this purpose, the base frame 12 comprises two opposite base frame openings 16, through which the pitch axis 15 extends. Specifically, the base frame openings 16 may include a bearing, in particular a rolling bearing, for mounting the pitch axis 15. The pitch axis thus rests on the base frame 12. In other words, by connecting the base frame and the pitch frame via the pitch axis, a relative tilting of the base frame 12 and the pitch frame 13 with respect to each other is possible. This relative movement enables the pitch movement of the patient couch 2.

The roll-pitch assembly 11 comprises a roll frame 14, which is essentially shaped like a frame and is essentially trapezoidal or rectangular in a top view. The roll frame 14 has a support section 18 on its upper side for the couch board section 7. The roll frame 14 at least partially surrounds the pitch frame 13. The roll frame 14 is pivotable and/or rotatable about a roll axis 17.

The roll axis 17 is essentially a mechanical rotation axis and is oriented in the longitudinal direction of the patient couch. The roll axis 17 rests on the pitch frame 13. In particular, the roll axis 17 extends through an opening 19 in the pitch frame. Here, the axis may be mounted via a bearing. Through this arrangement, the roll axis is also tilted during a pitch movement of the pitch frame 13, i.e., the roll axis 17 follows the pitch movement. To prevent the ingress of dirt and liquids, as well as to prevent hands or objects from reaching in and getting pinched, the roll-pitch assembly 11 is surrounded by a cover unit 19.

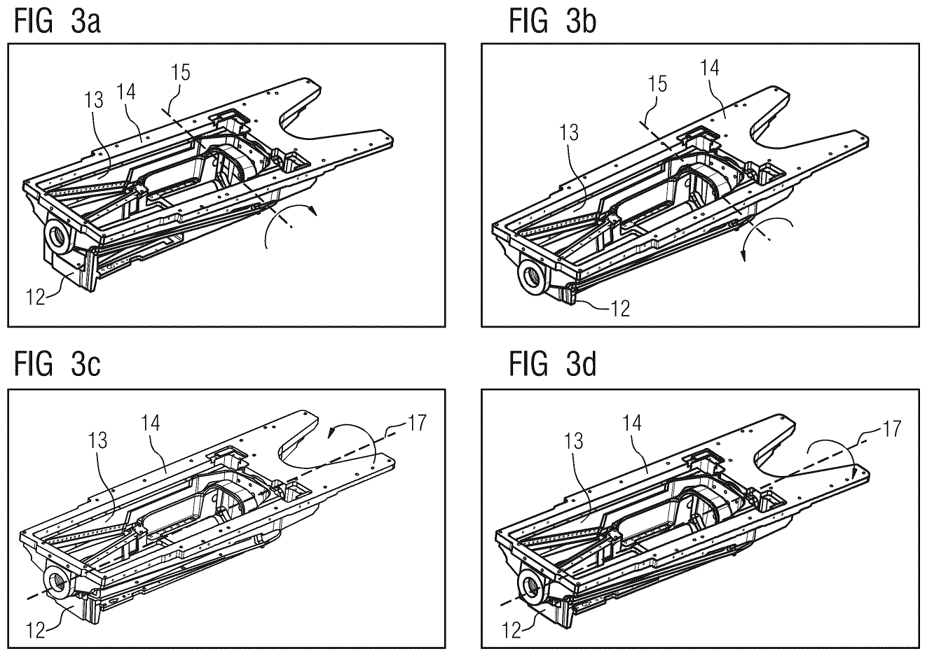

FIG. 3a depicts the pitch frame 13 and roll frame 14 in a pitched backward position, also called head-down position, showing the rear segment inclined downward and the front segment tilted upward. Consequently, the roll axis 17 tilts from the front-top to the rear-bottom.

FIG. 3b demonstrates the same frames 12-14 pitched forward, also calles head-up position, with the front parts tilted downwards and the rear sections upwards. The roll axis 17 inclines from the front-bottom to the rear-top.

FIG. 3c shows a leftward roll movement, with the left side of the roll frame 14 tilting downwards while the right side tilts upwards.

FIG. 3d depicts a rightward roll movement, showing the right side of the roll frame 14 tilting downwards and the left side upwards. The roll-pitch assembly 11, covered by the cover unit 19, is shown adapting to these movements.

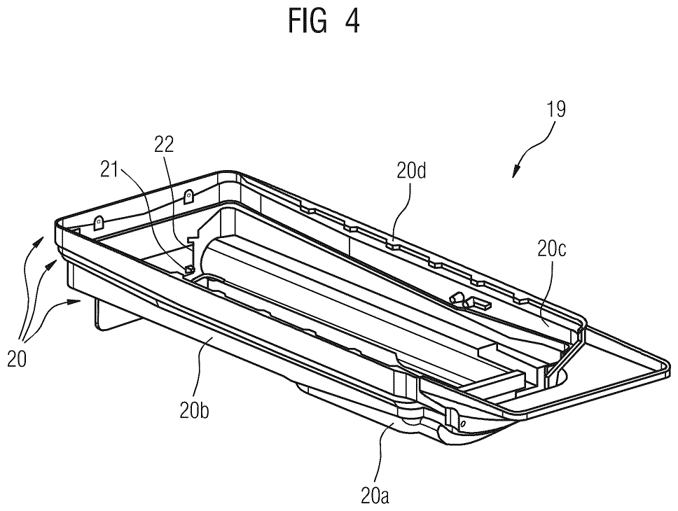

FIG. 4 illustrates an exemplary embodiment of the cover unit 19, which surrounds the roll-pitch assembly 11, particularly from the sides. The cover unit 19 connects to the base section 8 and the couch board section 7, forming a protective housing made up of multiple cover shells 20. These cover shells 20, made from rigid plastic, are telescopically connected, allowing for relative vertical movement.

The base cover shell 20a is mechanically attached to the base frame 12, while the pitch cover shell 20b, mechanically connected to the base cover shell 20a, features a projection 21 fitting into a receiving section 22 of the base cover shell 20a, allowing limited movement while ensuring a secure fit.

An intermediate cover shell 20c, telescopically connected to and/or interacting with both the pitch cover shell 20b and the roll cover shell 20d, enables seamless movement. The intermediate cover shell 20c is mechanically attached to the pitch frame 13.

The roll cover shell 20d, telescopically connected to the intermediate cover shell 20c, ensuring secure movement.

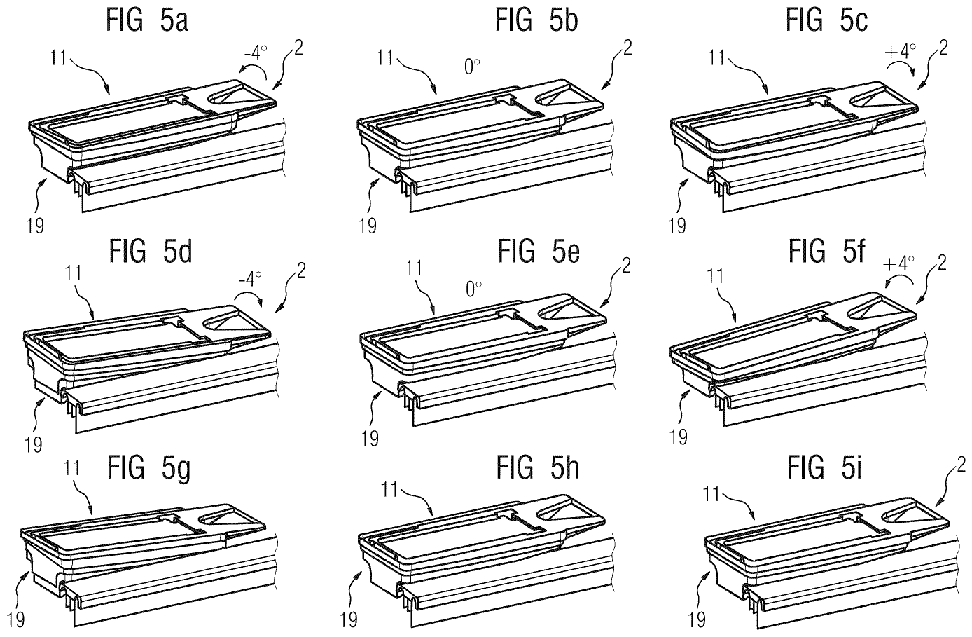

FIGS. 5a-c show the section for various roll movements. 5b illustrates the neutral position at 0° roll, 5 a depicts a 4° backward roll, and 5 c shows a 4° forward roll. During these movements, the roll cover shell 20d adapts by receiving or extending the intermediate cover shell 20c.

FIGS. 5d-f display the section for various pitch movements. 5e shows the neutral position at 0°pitch, 5d depicts a 4° rightward pitch also called head-down, and 5f shows a 4° leftward pitch also called head-up. The pitch cover shell 20b adapts similarly to the base cover shell 20a.

FIGS. 5g-i depict combinations of roll and pitch movements. 5h shows the neutral position at 0° roll and pitch, 5g illustrates a 4° rightward pitch also called head-down and 4° backward roll also called head-up, and 5i shows a 4° leftward pitch and 4° forward roll. The telescopically nested shells of the cover unit 19 effectively adapt to these combined movements, ensuring the mechanical parts remain covered and protected.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A patient couch for at least one of positioning or supporting a patient for radiotherapy, comprising:
a couch board section to accommodate the patient;
a base section attachable to a floor;
a roll-pitch assembly between the base section and the couch board section, the roll-pitch assembly configured to pivot the couch board section about at least one of a roll axis or a pitch axis; and
a cover unit forming a shell cover and comprising at least two cover shells, wherein one of the at least two cover shells forms a roll cover shell and another of the at least two cover shells forms a pitch cover shell, the roll cover shell is configured to compensate a roll movement of the roll-pitch assembly, and the pitch cover shell is configured to compensate a pitch movement of the roll-pitch assembly.

2. The patient couch of claim 1, wherein the at least two cover shells are movable relative to each other in a vertical direction.

3. The patient couch of claim 1, wherein at least one of
the at least two cover shells are slidable into and out of each other to compensate for at least one of the pitch movement or the roll movement, or
the at least two cover shells are receivable into each other to compensate for at least one of the pitch movement or the roll movement.

4. The patient couch of claim 2, wherein
one of the at least two cover shells includes a projection, and
another cover shell that is received by or receives the cover shell has a receiving section corresponding to the projection, wherein the projection and the receiving section form at least one of a guidance system or an interlock to limit a vertical movement.

5. The patient couch of claim 1, wherein one of the at least two cover shells forms an intermediate cover shell, the intermediate cover shell is between the roll cover shell and the pitch cover shell, and the intermediate cover shell is configured to compensate a roll movement and a pitch movement at least partially.

6. The patient couch of claim 1, wherein the roll-pitch assembly comprises,
a base frame, and
a pitch frame, the base frame at least one of (i) is fixedly connected to the base section or (ii) rests on the base section, the pitch frame is pivotable about the pitch axis, and at least one of the pitch axis rests on the base frame or the pitch axis is guided through an opening of the base frame.

7. The patient couch of claim 6, wherein the roll-pitch assembly further comprises,
a roll frame, the roll frame and the roll axis are pivotable, the roll axis at least one of rests on the pitch frame or is guided through an opening in the pitch frame such that the roll axis follows a pivoting of the pitch frame about the pitch axis.

8. The patient couch of claim 1, wherein a mechanical connection of the cover unit is made via a top cover shell and a bottom cover shell in a vertical direction, the top cover shell is mechanically connected to the couch board section and the bottom cover shell is mechanically connected to the base section.

9. The patient couch of claim 1, wherein the at least two cover shells are rigid.

10. A radiotherapy device comprising:
the patient couch of claim 1;
a controller configured to control or adjust the roll-pitch assembly; and
a linear accelerator or a radiation source configured to generate therapeutic radiation, wherein the patient couch is configured to position the patient can be positioned in a desired irradiation angle.

11. The patient couch of claim 2, wherein at least one of
the at least two cover shells are slidable into and out of each other to compensate for at least one of the pitch movement or the roll movement, or
the at least two cover shells are receivable into each other to compensate for at least one of the pitch movement or the roll movement.

12. The patient couch of claim 11, wherein
one of the at least two cover shells includes a projection, and
another cover shell that is received by or receives the cover shell has a receiving section corresponding to the projection, wherein the projection and the receiving section form at least one of a guidance system or an interlock to limit a vertical movement.

13. The patient couch of claim 12, wherein one of the at least two cover shells forms an intermediate cover shell, the intermediate cover shell is between the roll cover shell and the pitch cover shell, and the intermediate cover shell is configured to compensate a roll movement and a pitch movement at least partially.

14. The patient couch of claim 13, wherein the roll-pitch assembly comprises,
a base frame, and
a pitch frame, the base frame at least one of (i) is fixedly connected to the base section or (ii) rests on the base section, the pitch frame is pivotable about the pitch axis, and at least one of the pitch axis rests on the base frame or the pitch axis is guided through an opening of the base frame.

15. The patient couch of claim 14, wherein a mechanical connection of the cover unit is made via a top cover shell and a bottom cover shell in a vertical direction, the top cover shell is mechanically connected to the couch board section and the bottom cover shell is mechanically connected to the base section.
